(19) United States
(12) Patent Application Publication
(10) Pub. No.: US 2022/0171023 A1
(43) Pub. Date: Jun. 2, 2022

(54) APPARATUS AND METHOD FOR ESTIMATING HUMAN POSTURE

(71) Applicant: Research & Business Foundation Sungkyunkwan University, Suwon-si (KR)

(72) Inventors: Yu Sung KIM, Suwon-si (KR); Keun Hong CHAE, Suwon-si (KR); Seung Hyun KIM, Suwon-si (KR); Su Bin LIM, Suwon-si (KR)

(73) Assignee: Research & Business Foundation Sungkyunkwan University, Suwon-si (KR)

(21) Appl. No.: 17/535,837

(22) Filed: Nov. 26, 2021

(30) Foreign Application Priority Data
Nov. 27, 2020 (KR) 10-2020-0163216

(51) Int. Cl. G01S 7/41; G01S 13/02

(52) U.S. Cl. CPC G01S 7/412; G01S 13/0209; G01S 7/417

(57) ABSTRACT

The human posture estimating method according to the present disclosure includes transmitting and receiving a broadband or ultra-wideband wireless signal, allowing the received wireless signal to pass through a multi narrowband filter to be filtered into a plurality of frequency bands, and estimating a human posture by associating the filtered signal with human posture reference data acquired by machine learning and accurately estimates the human posture in an invisible region. Further, the transceiver is integrally formed to be useful in terms of the utilizability.

### TECHNICAL FIELD

The present disclosure relates to an apparatus and a method for estimating a human posture, and more particularly, to an apparatus and a method for deriving a human posture estimation result using a broadband or ultra-wideband wireless signal based on machine learning.

### BACKGROUND ART

In a computer vision field, as a technique of estimating a human posture, a method of estimating a human posture from an image has been developed in an early stage, but the image-based method has problems in that when the image is not properly formed due to the darkness of the space or an entire shape of the human body is not properly shown due to an obstacle in the space, the posture cannot be estimated and a picture of the human rather than an actual human is erroneously recognized to provide a wrong result.

A human posture estimation technique based on a wireless transceiver which has been developed to solve this problem solved the problem of the image-based technique, but the inventions in the early stage had a limitation in that sensors were attached to each part of the human body to use signals received by the sensors, so that there was a limitation in terms of usability that could not cope with situations that could occur intermittently, such as lifesaving, military, security, and disasters.

Thereafter, even though a technique of estimating a human posture by a signal which is directly reflected from the human by utilizing a frequency modulated continuous wave (FMCW) method has been developed and a technique using Wi-Fi signal has been also invented, there were problems such as a high cost, the need to install a transceiver in advance, and limitations in the usability of wireless signal characteristics due to the use of narrow bands.

### DISCLOSURE

### Technical Problem

In order to solve the above-described problems, an object of the present disclosure is to provide a human body estimating apparatus which is capable of estimating a position and a posture of a human body in a space including an invisible region using a broadband or ultra-wideband.

### Technical Solution

In order to achieve the above-described object, according to an aspect of the present disclosure, a human posture estimating method includes transmitting and receiving a broadband or ultra-wideband wireless signal, allowing the received wireless signal to pass through a multi narrowband filter to be filtered into a plurality of frequency bands, and estimating a human posture by associating the filtered signal with human posture reference data acquired by machine learning.

According to an exemplary embodiment, prior to the estimating of a human posture, the human posture estimating method further includes performing time band sampling on the wireless signal; and collecting the filtered frequency domain data and the time band sampling data.

According to an exemplary embodiment, the human posture reference data includes a plurality of feature points for each body part for every posture.

According to an exemplary embodiment, the human posture estimating method further includes performing machine learning by applying a wireless signal and human posture information synchronized with the wireless signal to a convolutional neural network.

According to an exemplary embodiment, in the performing of machine learning, human posture information is acquired based on the plurality of feature point data for each part of the human body acquired from an image which is photographed by a camera.

In order to achieve the above-described object, according to another aspect of the present disclosure, a human posture estimating apparatus includes a transceiver which transmits and receives a broadband or ultra-wideband wireless signal; a multi narrowband filter which allows the received wireless signal to pass through the multi narrowband filter to be filtered into a plurality of frequency bands; and a posture estimating unit which estimates a human posture by associating the filtered signal with human posture reference data acquired by machine learning.

According to an exemplary embodiment, the human posture estimating apparatus further includes a time band sampling unit which performs time band sampling on the wireless signal; and a pre-processing unit which collects the filtered frequency domain data and the time band sampling data.

According to an exemplary embodiment, the human posture estimating apparatus further includes a machine learning unit which applies a wireless signal and human posture information synchronized with the wireless signal to a convolutional neural network to perform machine learning.

According to an exemplary embodiment, the machine learning unit acquires human posture information based on the plurality of feature point data for each part of the human body acquired from an image which is photographed by a camera.

According to an exemplary embodiment, the time band sampling unit replaces some samples to remove strong reflection due to an obstacle.

### Advantageous Effects

The apparatus and the method for estimating a human posture according to the exemplary embodiment of the present disclosure do not have the limitation of a blocking phenomenon that the image is blocked, as compared with the camera-based position estimation of the related art and do not require an additional sensor and improve the position estimation performance by utilizing the broadband, as compared with the wireless signal-based position estimation of the related art.

The apparatus and the method for estimating a human posture according to the exemplary embodiment of the present disclosure use a transceiver which is integrated using the ultra-wideband so that the installation is easy. Further, a feature point of the human body through the ultra-wideband wireless signal and the machine learning is used so that the posture of the human body in an invisible region may be accurately estimated.

### DESCRIPTION OF DRAWINGS

FIG. 1 is a block diagram for explaining a configuration of a human posture estimating apparatus according to an exemplary embodiment of the present disclosure.

FIG. 2 is a flowchart for explaining a human posture estimating method according to an exemplary embodiment of the present disclosure.

FIG. 3 is a block diagram for explaining a configuration of a human posture estimating apparatus according to another exemplary embodiment of the present disclosure.

FIG. 4 is a flowchart for explaining a human posture estimating method according to another exemplary embodiment of the present disclosure.

FIG. 5 is a view illustrating a human posture estimating result according to an exemplary embodiment of the present disclosure.

### DETAILED DESCRIPTION OF THE PREFERRED EMBODIMENT

Those skilled in the art may make various modifications to the present disclosure and the present disclosure may have various embodiments thereof, and thus specific embodiments will be illustrated in the drawings and described in detail in the detailed description. However, this does not limit the present disclosure within specific exemplary embodiments, and it should be understood that the present disclosure covers all the modifications, equivalents and replacements within the spirit and technical scope of the present disclosure. In the description of respective drawings, similar reference numerals designate similar elements.

It should be understood that, when it is described that an element is “coupled” or “connected” to another element, the element may be directly coupled or directly connected to the other element or coupled or connected to the other element through a third element. In contrast, when it is described that an element is “directly coupled” or “directly connected” to another element, it should be understood that no element is present therebetween.

Terms used in the present application are used only to describe a specific exemplary embodiment, but are not intended to limit the present invention. A singular form may include a plural form if there is no clearly opposite meaning in the context. In the present application, it should be understood that the term “include” or “have” indicates that a feature, a number, a step, an operation, a component, a part or the combination thoseof described in the specification is present, but do not exclude a possibility of presence or addition of one or more other features, numbers, steps, operations, components, parts or combinations, in advance.

If it is not contrarily defined, all terms used herein including technological or scientific terms have the same meaning as those generally understood by a person with ordinary skill in the art. Terms defined in a generally used dictionary shall be construed that they have meanings matching those in the context of a related art, and shall not be construed in ideal or excessively formal meanings unless they are clearly defined in the present application.

Hereinafter, an exemplary embodiment of the present disclosure will be described in detail with reference to the accompanying drawings.

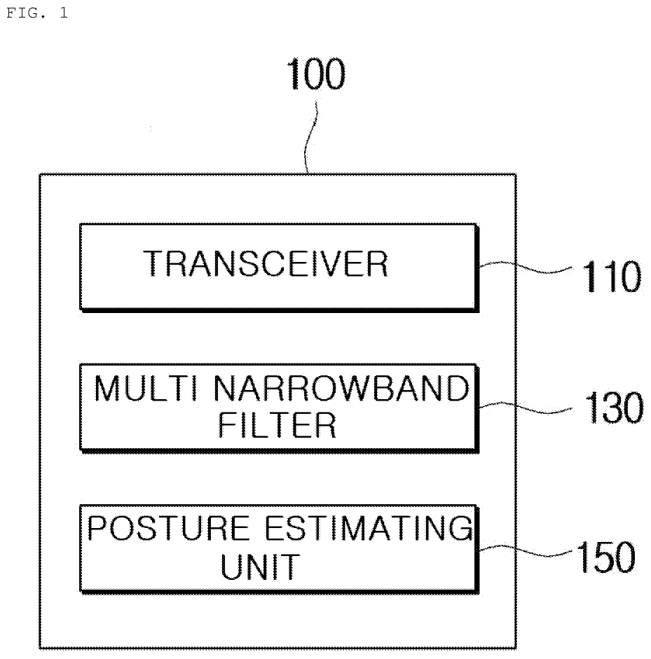

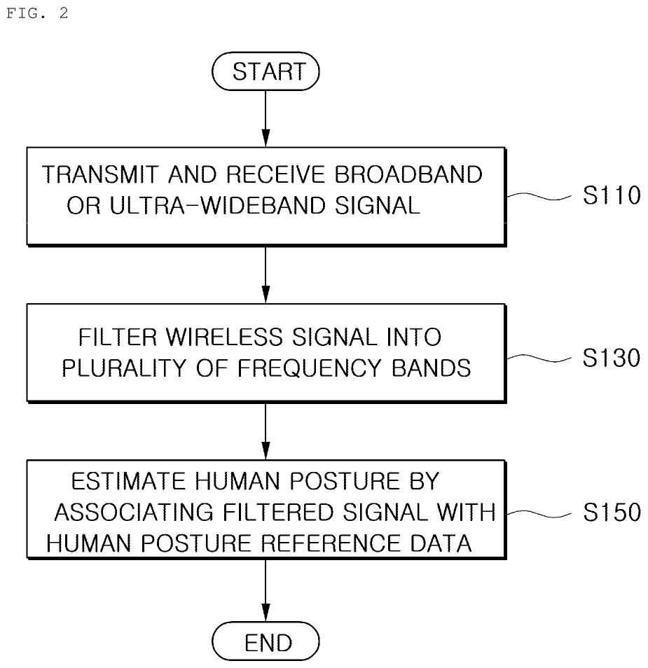

FIG. 1 is a block diagram for explaining a configuration of a human posture estimating apparatus according to an exemplary embodiment of the present disclosure and FIG. 2 is a flowchart for explaining a human posture estimating method according to an exemplary embodiment of the present disclosure.

As illustrated in FIG. 1, the human posture estimating apparatus 100 may include a transceiver 110, a multi narrowband filter 130, and a posture estimating unit 150.

In step S110, the transceiver 110 transmits and receives a broadband or ultra-wideband wireless signal.

According to an exemplary embodiment, the transceiver 110 may transmit and receive a UWB signal.

The UWB signal refers to a system which occupies an occupied bandwidth of 20% or more of a center frequency or a wireless transmission technique which occupies an occupied bandwidth of 500 MHz or higher and has a characteristic of transmitting a signal in a wide frequency band of 3.1 GHz or higher in a short time.

The signal transmitted by the transceiver 110 changes its property while being reflected, diffracted, and transmitted in a space and a human body and a signal which carries the corresponding property is received by the transceiver 110. Further, according to the exemplary embodiment, in order to acquire a spatial diversity, a plurality of transceiver pairs may be configured. In the exemplary embodiment, an example that uses first, second, and third transmitters and first, second, and third receivers has been assumed, but the number and the name of the transceivers may vary.

Signals in every band show different optical properties depending on a medium, for example, transmitting, diffracting, and reflecting properties are different. Therefore, when the wide bands can be utilized, various media information can be checked so that it means that more information for estimating the human posture can be utilized.

In step S130, the multi narrowband filter 130 allows the received wireless signal to pass through a multi narrowband filter to filter the signal into a plurality of frequency bands.

The multi narrowband filter 130 filters the broadband or ultra-wideband signal into a plurality of partial bands to utilize various bands having different physical properties. The multi narrowband filter 130 may be implemented by mounting a plurality of filters with different bands in parallel.

Further, according to an exemplary embodiment, the multi narrowband filter 130 may be manufactured to be integrated with the transceiver 110, but is not limited thereto.

In step S150, the posture estimating unit 150 associates the filtered signal with human posture reference data acquired by the machine learning to estimate a human posture.

The human posture reference data includes human posture information matching a wireless signal characteristic.

The human posture estimating apparatus according to the exemplary embodiment uses a broadband or ultra-wideband signal so that as compared with an example using an FMCW signal or a Wi-Fi signal, a plurality of frequency bands is simultaneously used so that an amount of available information in the same reception time is exceptionally large.

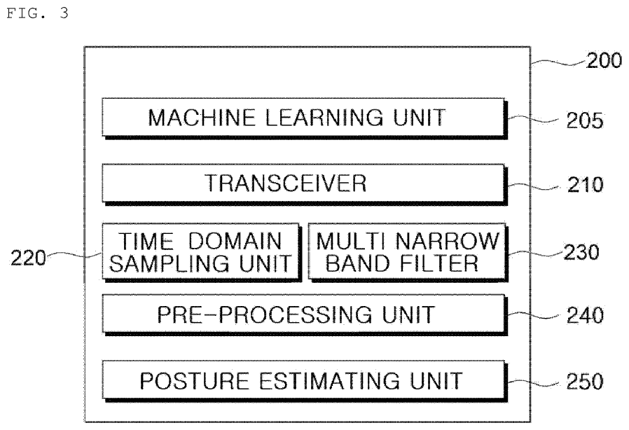

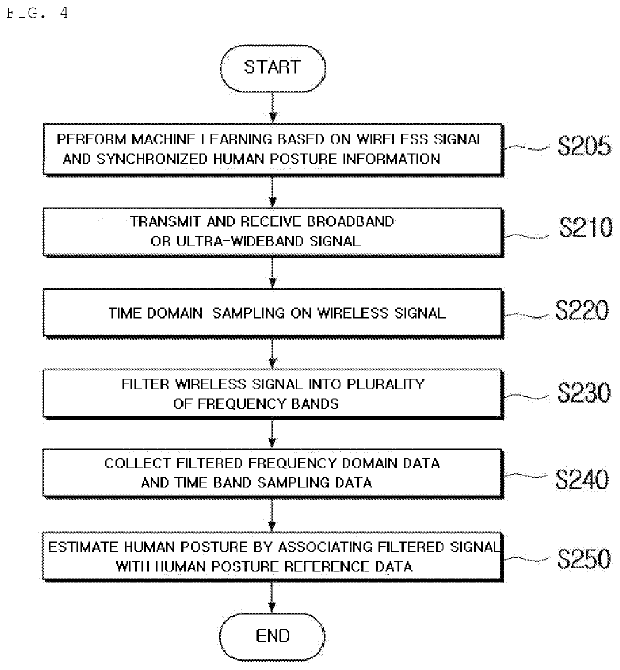

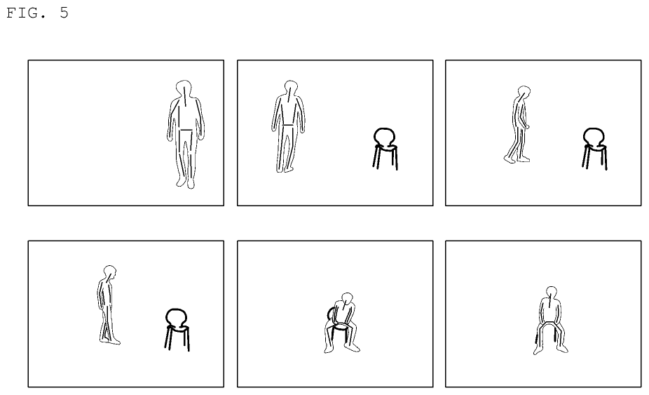

FIG. 3 is a block diagram for explaining a configuration of a human posture estimating apparatus according to another exemplary embodiment of the present disclosure and FIG. 4 is a flowchart for explaining a human posture estimating method according to another exemplary embodiment of the present disclosure. FIG. 5 is a view illustrating a human posture estimating result according to an exemplary embodiment of the present disclosure.

As illustrated in FIG. 3, a human posture estimating apparatus 200 may include a machine learning unit 205, a transceiver 210, a time band sampling unit 220, a multi narrowband filter 230, a pre-processing unit 240, and a posture estimating unit 250. The transceiver 210, the multi narrowband filter 230, and the posture estimating unit 250 are components corresponding to the transceiver 110, the multi narrowband filter 130, and the posture estimating unit 150 of FIG. 1 and may perform the functions of the corresponding components of FIG. 1. Hereinafter, a configuration differentiated from FIG. 1 will be mainly described.

Individual components which are separated in the exemplary embodiment of the present disclosure may be implemented as a combined physical component. For example, the time band sampling unit 220 and the multi narrowband filter 230 may be implemented by one wireless signal processing module.

Referring to FIGS. 3 and 4, in step S210, the transceiver 210 transmits and receives a broadband or ultra-wideband wireless signal.

According to the exemplary embodiment, in step S205, the machine learning unit 205 applies a wireless signal and human posture information which is synchronized with the wireless signal to a predetermined algorithm to perform the machine learning. The algorithm may be based on a convolutional neural network.

According to the exemplary embodiment, the machine learning unit 205 may acquire human posture information based on a plurality of feature point data for each part of the human body acquired from an image which is photographed by a camera.

The machine learning unit 205 performs the learning based on an image or spatial coordinate information which is synchronized with a wireless signal to be collected. To this end, groundtruth data which trains a neural network which processes an RF signal is required. The image or the spatial coordinate information may be acquired through a system implemented based on one or more cameras. The groundtruth data may include feature points for 13 body parts of the human posture.

According to the exemplary embodiment, the machine learning unit 205 finally outputs a heatmap on which a prediction probability for 13 body parts of the human posture is represented on a 2D spatial coordinate, with respect to the image collected through one or more cameras, to use this as human posture reference data.

In step S210, the transceiver 210 may transmit and receive a broadband or ultra-wideband signal. The transceiver 210 transmits the broadband or ultra-wideband signal to a section of interest and then receives a signal which is transmitted, diffracted, and reflected by different media in the section of interest.

The transceiver 210 is implemented as one component so that it is advantageous for availability and portability.

In step S220, the time band sampling unit 220 performs the time band sampling on the wireless signal.

The time band sampling unit 220 allows the wireless signal to be received at different timings according to (TOF) a time of flight of the transmitted wireless signal. That is, the signals at different timings have different spatial information. A signal collected for every time slot is separately processed for the collected signal so that the signal for every time slot is utilized to estimate the human posture, rather than a synchronized correlation result of the received signal which is generally utilized.

According to the exemplary embodiment, the time band sampling unit 220 may replace some samples to remove strong reflection due to the obstacle. By doing this, the accuracy for estimating a human estimation posture may be improved.

In step S230, the multi narrowband filter 230 allows the received wireless signal to pass through a multi narrowband filter to filter the signal into a plurality of frequency bands.

The multi narrowband filter 230 filters the broadband or ultra-wideband signal into a plurality of partial bands to utilize various bands having different physical properties. The multi narrowband filter 130 may be implemented by mounting a plurality of filters with different bands in parallel.

Further, according to the exemplary embodiment, the multi narrowband filter 230 may be manufactured to be integrated with the transceiver 210, but is not limited thereto.

The step S220 and the step S230 may be performed in a switched order or performed in parallel. According to another modified example, any one of the step S220 and the step S230 may be omitted.

In step S240, the pre-processing unit 240 collects filtered frequency domain data and time band sampling data.

According to the exemplary embodiment, the pre-processing unit 240 may collect different types of data by two-dimensionally rearranging the plurality of data in the frequency domain and the plurality of sampling data in the time band.

In step S250, the posture estimating unit 250 associates the filtered signal with human posture reference data acquired by the machine learning to estimate a human posture.

According to the exemplary embodiment, the posture estimating unit 250 associates the filtered signal with the human posture reference data acquired by the machine learning to derive a human body feature point heatmap as illustrated in FIG. 5.

The human posture estimating apparatus according to the exemplary embodiment of the present disclosure may be utilized to identify a human and estimate a position in a specific space and as the posture estimation field, it may be utilized in various fields such as games, medical cares, disasters, firefighting, security, and military.

The human posture estimating apparatus according to the exemplary embodiment of the present disclosure applies machine learning to significantly improve the speed and the accuracy of estimating the human posture.

The human posture estimating method according to the exemplary embodiment of the present invention may be implemented as a program command which may be executed by various computers to be recorded in a computer readable medium. The computer readable medium may include solely a program command, a data file, and a data structure or a combination thereof. The program commands recorded in the computer readable medium may be specifically designed and constructed for the present invention or those known to those skilled in the art of a computer software to be used. Examples of the computer readable recording medium include magnetic media such as a hard disk, a floppy disk, and a magnetic tape, optical media such as a CD-ROM or a DVD, magneto-optical media such as a floptical disk, and a hardware device which is specifically configured to store and execute the program command such as a ROM, a RAM, and a flash memory. Further, the above-described medium may be a transmission medium such as optical or metal wire or a waveguide including a carrier wave which transmits a signal specifying a program commands or data structures. Examples of the program command include not only a machine language code which is created by a compiler but also a high level language code which may be executed by a computer using an interpreter. The above-described hardware device may be configured to operate as one or more software modules in order to perform the operation of the present invention and vice versa.

It will be appreciated that various exemplary embodiments of the present disclosure have been described herein for purposes of illustration, and that various modifications, changes, and substitutions may be made by those skilled in the art without departing from the scope and spirit of the present disclosure. Accordingly, the exemplary embodiments carried out in the present disclosure are intended to not limit but describe the technical spirit of the present disclosure and the scope of the technical spirit of the present disclosure is not restricted by the exemplary embodiments. The protective scope of the present disclosure should be construed based on the following claims, and all the technical concepts in the equivalent scope thereof should be construed as falling within the scope of the present disclosure.

### DESCRIPTION OF MAIN REFERENCE NUMERALS OF DRAWINGS

100, 200: Human posture estimating apparatus

205: Machine learning unit

110, 210: Transceiver

220: Time band sampling unit

130, 230: Multi narrowband filter

240: Pre-processing unit

150, 250: Posture estimating unit

1. A human posture estimating method, comprising:
transmitting and receiving a broadband or ultra-wideband wireless signal;
allowing the received wireless signal to pass through a multi narrowband filter to be filtered into a plurality of frequency bands; and
estimating a human posture by associating the filtered signal with human posture reference data acquired by machine learning.

2. The human posture estimating method according to claim 1, further comprising:
prior to the estimating of a human posture, performing time band sampling on the wireless signal; and
collecting the filtered frequency domain data and the time band sampling data.

3. The human posture estimating method according to claim 1, wherein the human posture reference data includes a plurality of feature points for each body part for every posture.

4. The human posture estimating method according to claim 1, further comprising:
performing machine learning by applying a wireless signal and human posture information synchronized with the wireless signal to a convolutional neural network.

5. The human posture estimating method according to claim 4, wherein in the performing of machine learning, human posture information is acquired based on a plurality of feature point data for each part of the human body acquired from an image which is photographed by a camera.

6. A human posture estimating apparatus, comprising:
a transceiver which transmits and receives a broadband or ultra-wideband wireless signal;
a multi narrowband filter which allows the received wireless signal to pass through the multi narrowband filter to be filtered into a plurality of frequency bands; and
a posture estimating unit which estimates a human posture by associating the filtered signal with human posture reference data acquired by machine learning.

7. The human posture estimating apparatus according to claim 6, further comprising:
a time band sampling unit which performs time band sampling on the wireless signal; and
a pre-processing unit which collects the filtered frequency domain data and the time band sampling data.

8. The human posture estimating apparatus according to claim 6, wherein the human posture reference data includes a plurality of feature points for each body part.

9. The human posture estimating apparatus according to claim 6, further comprising:
a machine learning unit which applies a wireless signal and human posture information synchronized with the wireless signal to a convolutional neural network to perform machine learning.

10. The human posture estimating apparatus according to claim 9, wherein the machine learning unit acquires human posture information based on a plurality of feature point data for each part of the human body acquired from an image which is photographed by a camera.

11. The human posture estimating apparatus according to claim 7, wherein the time band sampling unit replaces some samples to remove strong reflection due to an obstacle.
